(19) United States
(12) Patent Application Publication
(10) Pub. No.: US 2015/0273221 A1
(43) Pub. Date: Oct. 1, 2015

(54) METHOD AND APPARATUS FOR INDUCING ENDOGENOUS GENERATION OF ADULT STEM CELLS

(71) Applicant: Electrohealing Holdings, Inc., Estero, FL (US)

(72) Inventors: Michael R. Manning, Langhome, PA (US); Michael Weiner, Estero, FL (US)

(21) Appl. No.: 14/673,012

(22) Filed: Mar. 30, 2015

Related U.S. Application Data
(60) Provisional application No. 61/971,870, filed on Mar. 28, 2014.

(51) Int. Cl. A61N 1/378; A61M 37/00; A61M 5/142; A61N 1/362; A61B 5/00

(52) U.S. Cl. CPC A61N 1/3787; A61N 1/362; A61B 5/00; A61M 5/142; A61M 37/00

(57) ABSTRACT

The present disclosure relates to methods and apparatus relates to the endogenous generation of stem cells. More specifically, the present disclosure describes an implantable device that may induce the dedifferentiation of nucleated cells into adult stem cells. In some aspects, the implantable device may further capture and deliver the generated stem cells to the target site for treatment.

### CROSS-REFERENCE TO RELATED APPLICATION

This application claims priority to and the full benefit of U.S. Provisional Patent Application Ser. No. 61/971,870, filed Mar. 28, 2014, and titled “Endogenous Stem Cell Generation, Capture, and Delivery of Patient Compatible Stem Cells”, the entire contents of which are incorporated herein by reference.

### FIELD OF THE DISCLOSURE

The present disclosure relates to methods and apparatus relates to the endogenous generation of stem cells. More specifically, the present disclosure describes an implantable device that may induce the dedifferentiation of nucleated cells into adult stem cells.

### BACKGROUND OF THE DISCLOSURE

Stem cell therapy has shown significant promise in addressing a number of maladies. Stem cells today work the best when injected into the myocardium. They have to be sourced for procedures that vary from getting them from bone marrow, or rendered from the patient's fat, or collected from a stored umbilical cord, or processed with an acidic shock, etc. Each takes time and bears a cost.

In many cases, transplanting or injecting cells may be a useful treatment method to repair damaged tissue. However, there is a risk that the body may reject the treatment. Such treatments are often time consuming, costly, expensive, and sometimes painful procedures such as bone marrow transplant, cutting off body fat to generate stem cells through a process that can take several weeks. In some cases, stem cells may be stored by saving a baby's umbilical cord and freezing it for years to come, something especially difficult for those who do not have the money or the foresight.

### SUMMARY OF THE DISCLOSURE

Accordingly, the present disclosure provides an effective way to treat patients with stem cells that does not require painful, time-consuming, and expensive procedures, and one that does not require the foresight of preserving an umbilical cord. In some embodiments, a pacemaker is equipped to offer a repair function via endogenous generation of stem cells in addition to the regulation of heartbeats.

Accordingly, the present disclosure relates to the endogenous generation of stem cells. More specifically, the present disclosure describes an implantable device that may induce the dedifferentiation of nucleated cells into adult stem cells.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates an exemplary implantable device, such as a pacemaker.

FIG. 2 illustrates an exemplary improved electrical lead for use in an implantable device.

FIG. 3 illustrates an exemplary embodiment of a data flow chart in an implantable device.

FIG. 4 illustrates exemplary method steps for inducing the endogenous generation of adult stem cells.

### DETAILED DESCRIPTION

The present disclosure provides generally for methods and apparatus facilitating endogenous generation of stem cells. More specifically, the present disclosure describes an implantable device that may induce the dedifferentiation of nucleated cells into adult stem cells.

In some embodiments, the implantable device may allow for the capturing a large supply of adult stem cells in support of any therapeutic intervention. As these are generated endogenously, the adult stem cells will not be rejected. In some aspects, the implantable device may deliver these cells to the target need. In some implementations, implanting the device, permanently or temporarily, may provide a constant flow of available cells to the target. In some aspects, the implantable device may comprise means of directing cells to specific targets, such as the skin of a patient suffering from endemic wound healing, or recovering from surgery.

In some aspects, a supply of stem cells may be useful to a human or animal for treatment of many medical conditions, such as spinal fusion, knee and other orthopedic needs (injected batter alternative), retina and ocular, bone repair, muscle repair, brain repair, nerve damage, sensory impairment, pediatric requirements, sickle cell anemia, other anemia. In some embodiments, stem cells may be useful in the regeneration of organs or tissue for implanting.

In the following sections, detailed descriptions of examples and methods of the disclosure will be given. The description of both preferred and alternative examples though through are exemplary only, and it is understood that to those skilled in the art that variations, modifications, and alterations may be apparent. The implantable device is not meant to be limited to any particular medical device, the function of inducing and increasing the supply of adult stem cells endogenously with our unique promoter can be applied into an endless number of embodiments beneficial to human and animal health. It is therefore to be understood that the examples do not limit the broadness of the aspects of the underlying disclosure as defined by the claims.

### GLOSSARY

Generator: as used herein refers to a device that may induce the endogenous generation of adult stem cells.
Implantable Device: as used herein refers to a device comprising a generator that may be permanently or temporarily implanted into the body, wherein the implanting may place the generator into contact with the induction site.
Induction Site: as used herein refers to a location on the body where a device may induce the endogenous generation of adult stem cells.
Target: as used herein refers to a location in the body that may benefit from exposure to adult stem cells.

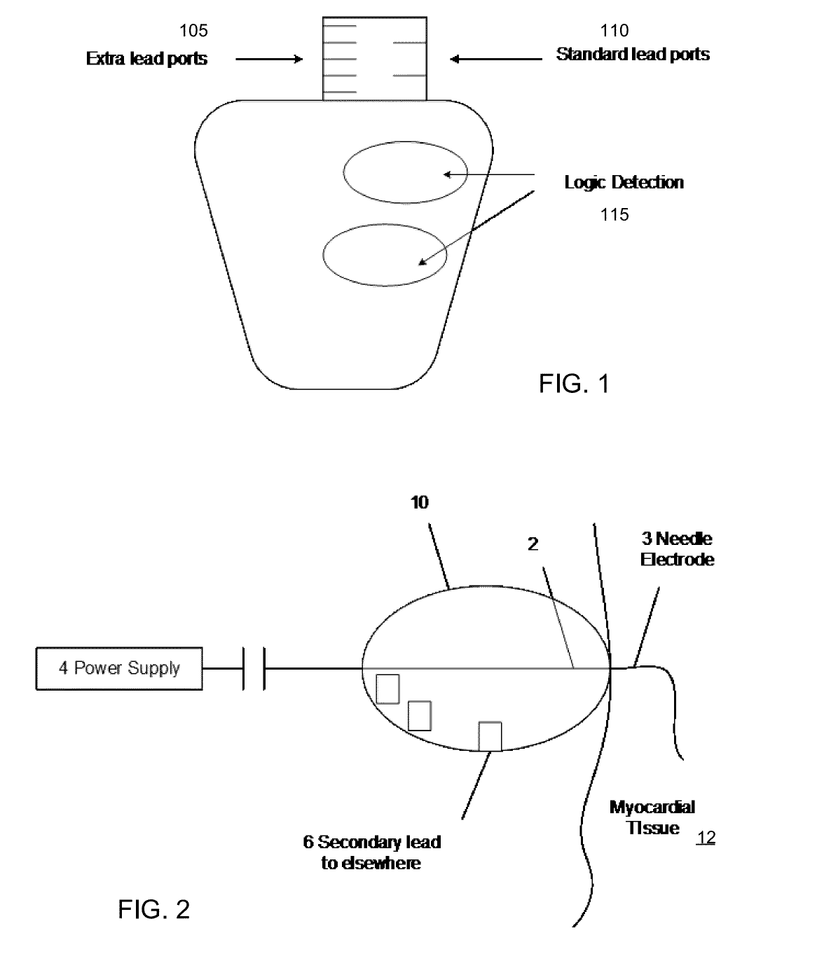

Referring now to FIG. 1, an exemplary implantable device, such as, in some exemplary embodiments, a pacemaker, is illustrated. In some aspects, the implantable device may comprise a general medical device with standard lead ports 110, which may be used for traditional medical device functions, such as heartrate monitoring or pace making. In some implementations, a general medical device may comprise extra lead ports 105, which may allow for an expanded range of uses for the device. In some embodiments, a generator may rely on the extra lead ports 105 without requiring extensive modifications. In some aspects, the implantable device may comprise one or more logic blocks 115, which may control the functions of the device. In some aspects, the logic blocks 115 may control the frequency of the electrical impulses. Logic blocks may be fashioned from one or both of: electronic circuitry and integrated circuits on silicone substrates. A power source is utilized to provide electrical power to the logic blocks 115. In some embodiments, the power source may comprise a solid-state battery.

Referring now to FIG. 2, an improved electrical lead that can be used both with pacemaker leads to perform an added function (stem cell generation) or can be used in this or other configurations to send stem cells to any place in the body that the lead may reach or be placed within, is illustrated. A stem cell generation device may include enhancing a multifunction device, such as a demand pacemaker device, a stem generation device may be dedicated or limited function device, including: stem cell generation, low voltage (under 5 volts vs 800 volts) defibrillation, insulin pump and combined pacing/stem cell generation/defibrillation.

In some embodiments, a single wire dual control or multiple control (low voltage defibrillation for V-FIB and A-FIB) system may be provided. An insulin pump may include stem cell generation circuitry and electro-stimulation leads whereby the system may be implanted or temporarily worn on the body or kept by a bed in which a patient rests. A power source 4 may include, by way of non-limiting example: be a continuous power supply, a battery, a rechargeable unit, a capacitor, bio-cell, n induction coil and associated electromagnetic wave, or other source of electrical power. In some embodiments, a power source may be provided by a pacemaker or another device that may have the ability to send power to the lead, optionally control the stem cell generation on or off, communicate with controllers outside a user's body.

In the some embodiments, electrical elements may be added to a pacemaker lead (ITEM I) whose ordinary function is to sense the heart signal and delivery pacing when needed. In some embodiments, the lead may go in the ventricle through the valve, and connect to the myocardium, wherein the lead may perform all normal pacemaker functions, and also may deliver low voltage defibrillation. In some aspects, the pacemaker may provide the Far Field Stimulation of a Poincare point.

A single lead electrode, such as a needle electrode 3, that can pace and sense the heart, and when needed, deliver a different voltage that can cause the stem cell generation process, by varying ac and dc currents. Far field stimulation of a Poincare Space device with multiple electrodes are used on the heart to bring the heart to homeostatis is the shortest possible time, in vivo. Each of these electrodes can also generate and deliver stem cells when indicated (by a sensor) are needed. This may enable adequate coverage across the heart to both treat heart disease electrically and also by generating and releasing stem cells to the heart, as part of a response to an MI or other cardio event.

In some implementations, a generator 10 may be interposed into the lead, wherein the generator 10 may comprise a casing made of material that will allow cells to flow in and out, but will not release silver ions into the blood. In some aspects, silver ions may be generated when the electrical coil is powered up, and the electrical current may cause silver ions to be released from the silver source, in the case a rod 2, into the casing, which has blood cells flowing through the device wall. The silver ions that are released may not exit the cell wall and may have an effect on a portion of the nucleated white blood cells in the blood to dedifferentiate into adult stem cells. Some of these cells may remain in the generator and others will flow out of the device wall, back into the blood supply, in the case in the myocardium.

In some aspects, some of the dedifferentiated cells will flow into the hollow point screw connector that connects the lead to the myocardium. The dedifferentiated cells may transmit sensing signals from the heart (to the pacemaker or other device) to the screw connector electrode, which is connected into the myocardium, where it can also deliver pacing signals and low voltage defibrillation. The screw connector is unique in that it has a hollow center in the 5 mm range, but may be larger or smaller. This electrode housing may be located in the ventricle and constantly in the blood stream. This may allow it to bring in white blood cells and convert them to dedifferentiated cells, which may be released back into the blood supply in the ventricle and/or into the myocardium through the hole in the middle of screw connector flowing right into the myocardial sub-surface tissue 12.

In some embodiments, the connector may be located in the blood supply within the heart ventricle, and the electrode generating case may be in blood, wherein the blood and dedifferentiated cells may remain present. Two components are depicted, the first is the alternative lead tips that may be inserted into the collector device and power other leads. This can be used to connect an additional lead to the system, and route those leads to the point of need for a dual function device to generate stem cells. These female receptors will accept the lead tips of additional generator devices, and route this lead to an area where stems cells are desired to flow in this case we will be able to route the lead to anywhere in the body where you want to deliver stem cells. This lead will pick up the supply of cells in the housing and deliver them to the intended point.

In some embodiments, the electrode may be part of a new and improved pacemaker. Upon detection that the heart is in an MI, it curtails pacing (to avoid putting the heart into fibrillation), and it induces a flow of stem cells into the heart chamber and blood supply. Since pacemakers are a well-developed source of implantable power, the pacemaker may supply power to multiple electrodes, such as secondary electrode 6, in places in a patient as needed. In some aspects, a stem cell eluting device may be directed to a target site, where a supply of adult stem cells may be needed.

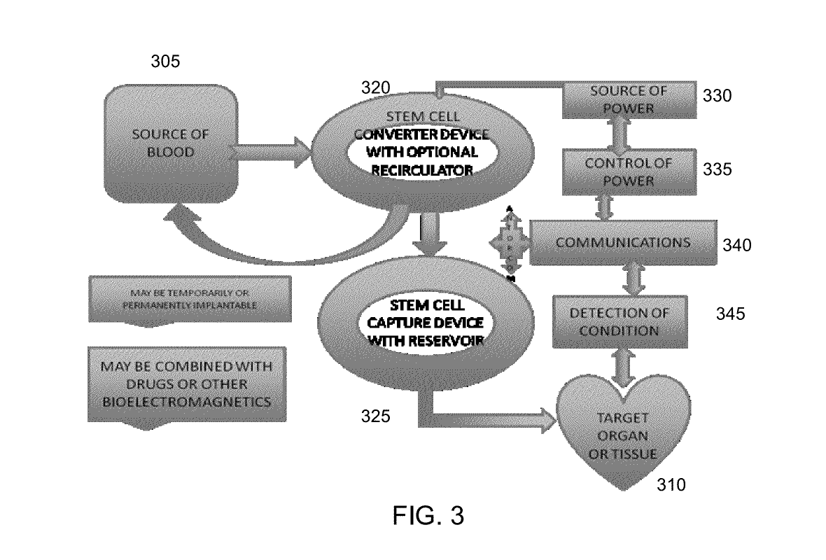

Referring now to FIG. 3, a flow chart illustrates data flow in an implantable device. In some aspects, an implantable device may comprise a power source 330 and a control logic block 335, which may drive the functionality of the implantable device. In some aspects, the implantable device may comprise a network access device 340 that may communicate with external devices, which may allow remote control of the implantable device.

In some implementations, the implantable device may comprise one or more sensors 345, which may be configured to monitor the conditions of one or both the induction site 305 or the target site 310. In some aspects, the implantable device may comprise a generator with optional recirculator 320, which may increase the efficiency of the induction. In some embodiments, the implantable device may comprise a capture mechanism 325 that may have a reservoir to hold stem cells. In some aspects, the reservoir may be removable and externally stored. In some aspects, the reservoir may be contained within the implantable device and may provide a replenishable source of stem cells, wherein the stem cells are generated via electrical stimulation via the stem cell generator and delivered into the.

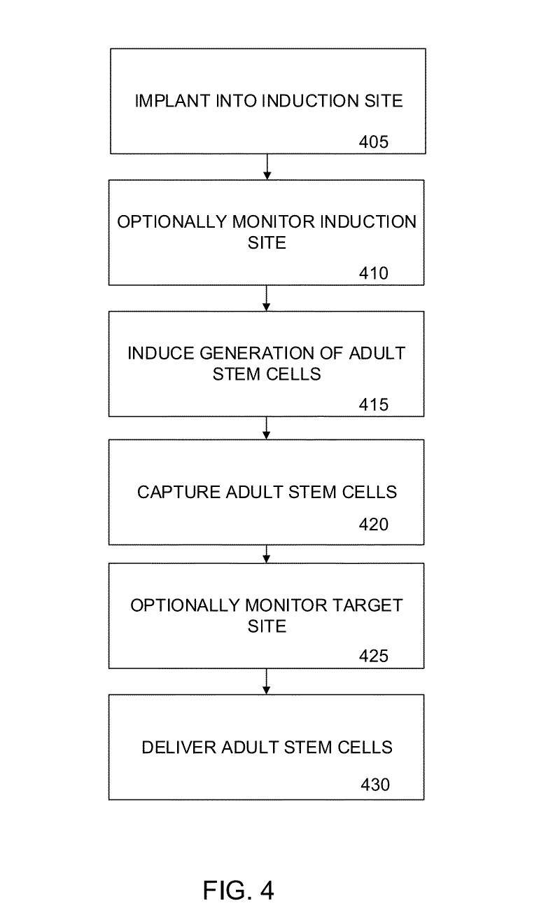

Referring now to FIG. 4, exemplary method steps for inducing the endogenous generation of adult stem cells are illustrated. In some embodiments, at 405, the implantable device may be implanted into an induction site. At 410, the implantable device may optionally monitor the condition of the induction site. For example, the implantable device may comprise a pacemaker, which may monitor conditions traditionally associated with a pacemaker. In some embodiments, the implantable device may monitor conditions that may affect the effectiveness of the induction, such as pH or concentration of nucleated cells. Additional monitoring may include almost any measurable biometric, such as, for example, one or more of: heart rate, blood pressure, blood glucose level, blood constituent level, white blood cell count, red blood cell count, enzyme levels, mineral levels, or other measurable biometric.

At 415, generation of adult stem cells may be induced, wherein nucleated cells are induced to dedifferentiate into adult stem cells. At 420, the adult stem cells may be captured. In some embodiments, the captured cells may be stored within the implantable device, which may be accessed when required, such as at 430. In some aspects, the captured cells may be collected in a separate receptacle that may be stored separately, such as at a hospital.

In some embodiments, at 425, the target site may be optionally monitored. In some aspects, the monitoring may detect when delivery of stem cells may be necessary, which may trigger a delivery, such as at 430. In some aspects, the monitoring may comprise supplemental or related condition monitoring. At 430, the adult stem cells may be delivered to the target site. In some aspects, the delivery may comprise a stem cell bath, target site injection, or other delivery means.

In some embodiments, a capture device that may capture adult stem cells flowing in blood, lymph, and other fluids, and store them, and/or deliver them, may be coupled or integrated with an implantable device that may induce the generation of an increased number of stem cells endogenously, in the body, in real time, by generating the conversion of nucleated cells in an organism to dedifferentiated adult stem cells.

In some aspects, a capture mechanism may comprise a cell scaffolding device for the purposes of growing tissue, with the addition of vibrations energy, which is designed as vibration up to PEMF and GHZ range mm-wave therapy, such as described in U.S. Pat. No. 6,995,013, hereby incorporated by reference.

In some aspects, the implantable device can be paired with other functions for emergency use in hospitals, ambulances, MASH units, research laboratories, extended care facilities, organ growth, stem cell banks and facilities. It can be paired with temporary disposable catheters, including a temporary pacemaker defibrillator lead.

In some implementations, the implantable device may comprise a generator that may function as a standalone device that may increase the adult stem cells in a supply of blood or lymph by causing nucleated cells to dedifferentiate into adult stem cells, in a quantity substantially greater than normal. The generator may operate alone or in a combination of medical devices and available across a wide variety of indications. The system of induced generation, collection and storage may comprise a two part system.

In some aspects, the implantable device may comprise a pacemaker. A pacemaker is ordinarily used to detect and correct bradycardia, by speeding up a slow heart. There are many sophisticated designs such as dual chamber pacing, and others. Coupling the traditional function of a pacemaker with a generator may allow the implantable device to detect or assist a patient when they experience a myocardial infarction, or heart attack.

In some embodiments, the implantable device may sense indications of myocardial infarction or heart attack, and by issuing a signal to the pacemaker lead in the myocardium, the lead may induce generation of an army of adult cells and release them in the blood directly within the ventricle, and also into the myocardium via the tip of the screw into the myocardium. This may enable the pacemaker to increase the supply of the patient's own stem cells available to the body, including thousands more immediately available to the infarcted tissue through the body's normal repair system. This flooding of stem cells to the heart and the circulatory system may continue until the patient, who is also alerted they have experienced an MI (they are not always aware), may receive medical care from a professional.

In some aspects, the implantable device may store stem cells and keep them alive and vibrant and available for a cardiologist to use for these injections, and then the cardiologist may insert additional leads into the heart where they wish to see an increased supply. In some embodiments, attaching the leads to the pacemaker may provide an ongoing supply direct to one or more points with the heart, or on the outer surface, with optional endocardial leads.

In some aspects, the implantable device may comprise or may be coupled with a delivery mechanism, which may deliver adult stem cells to a target site. In some embodiments, the implantable device may comprise of may be coupled with a capture mechanism, which may collect and store generated adult stem cells. The stored generated adult stem cells may be delivered by the delivery mechanism or may be removed and stored for future use, such as by a physician, physical therapist, or treating cardiologist. The stem cells can be delivered via a flow to tissue, or a needle into tissue, or a device implanted into tissue to assist with engrafting, or other means.

In the heart there are constantly a flow of cells that may be directed or drawn to a spot by the body. By inducing a specific signal into a device, turning on an electrode with a second wire may cause direct a flow of dedifferentiated adult stem calls to the point of need.

The adult stem cell elution can be triggered automatically upon certain identified conditions of EGG. The specific reaction described below will cause a rapid flow of adult stem cells to flood the chamber of the directed target of the induced blood flow. The electrode, once activated, will start a conversion and elution process forming a high flow of adult stem cells to the wearer, assisting it in maintaining healthy cells.

A series of catheters may be preinstalled to enable stem cell infusion to parts of the myocardium, or directly into the blood within the myocardium. By increasing the number of adult stem cells circulating within the myocardium damaged tissue can get access to that many more stem cells per number of blood cells. Infusion of stem cells into the myocardium also increases the stem cells available to all organs and systems in the body to utilize.

In some aspects, the implantable device may regulate pH variance and electrical fields, which may provide the ability to commence stem cell flow whenever algorithms in the device indicate based on sensing conditions, and then turn on the generator controlled by an electrical signal that may be transmitted over the pacing lead wire without interruption of pacing. In some embodiments, the implantable device may be remotely controlled and programmed, allowing control of the stem cell generator; collector and administration systems from outside the body. Remote access and control may allow a physician to control and override the device.

In some implementations, the implantable device may signal the patient if the event, ensuring the patient realizes they have experienced an MI, as mild ones are often missed. A pacemaker trained cardiologist or intern may administer an injection of the patient's own stem cells directly into myocardial tissue using an incremental lead that may induce and collect stem cells that are flowed to the infarction tissue in real time, using the incremental function of the pacemaker and its ability to support and deploy additional leads. In some aspects, this may allow real time flow of stems cells into the myocardium controlled by the physician remotely, which may shorten the time to therapy and thus also reduce the progression to congestive heart failure (CHF) of the heart attack victim. In the case of a patient with an MI the availability of stem cells can reduce the damage of heart tissue as well as reducing the onset of CHF.

In some embodiments, generators may aid treatments of the knee, spine, liver, pancreas, and also increase the flow to cell scaffold devices growing cells both in-vivo, ex-vivo and in-situ. This function can also be added to ICD and neuro-stimulators, insulin and other drug pumps; and devices whose primary function is stem cell inducement through real time stem cell dedifferentiated cells working on the supply of white blood cells circulating in the body. In some aspects, the generator may control one or more electrical conditions, such as voltage, polarity, power and waveform, and pH conditions, or other methods of inducing dedifferentiation in a live cell, whether in-vivo, ex-vivo, in situ, or in an automated process.

A stem cell bioreactor may have the ability to maintain a proper salinity, pH level, temperature, dissolved carbon dioxide and oxygen levels in a growth medium along with the correct cell nutrients. It should be noted that sudden changes in ionic concentrations in the fluid around cells other than hydrogen ion concentration (pH) are effective as dedifferentiation triggers provided that the ions used are not poisonous to the cell. That is, ions of sodium, potassium, calcium, silver and magnesium may be employed whereas ions of barium, beryllium, antimony, thallium, mercury, copper and lead may not be used.

In some embodiments, the speed of a pH shift, rather than the total numbers of pH points shifted, is controlled. For example, a pH shift of one point in one second may be used to dedifferentiate. A pH shift of two points over 20 seconds, conversely, may not dedifferentiate.

In some aspects, inducing a very rapid spike in PH may induce dedifferentiation. This must be done within precise degrees of time and intensity. For example, a spike of less than one second may be effective, whereas a transition over 8 or 20 seconds will not. In some embodiments, combining inducing methods may enhance the quantity speed and optimal environment to generate these cells. In some aspects, the dedifferentiation process may be initiated in as little as 10 minutes, with a very low power signal.

In some embodiments, an implantable device, such as a pacemaker, may sense a myocardial infarction, and when detected, may send a signal along the lead to the pacing electrode, which may be located in the ventricle, ensconced in blood, to start the process of dedifferentiating white blood cells into adult stem cells. In some implementations, the cells may be directed into the ventricles to increase the body's natural method of uptake, and a delivery mechanism may drive stem cells directly into the myocardium through a needle like channel in the pacemaker screw, which may place stem cells into various depths of myocardial tissue.

In some aspects, the placement of the pacemaker lead may not be near the location of the MI, which now needs the influx of adult stem cells. The implanted device and the electrode can accommodate additional leads, which can be routed by a cardiologist to the myocardium needing the delivery of cells.

In some embodiments, the implantable device may comprise an electrode capable of generating and eluting stem cells from blood, a means of gaining access to the blood, and a means of outputting the adult stem cells back into the body. In some aspects, additional electrodes may be implanted during an ER treatment to all points of need, and, immediately upon detection of the MI the myocardium is awash in a hundred times greater supply of stem cells to tissue than the body is naturally capable.

A major risk associated with having an MI is the heart transitions to a state leading to congestive heart failure (CHF), which may cause a person to die within five years. In some aspects, an increase in normal stem cell production by the body by an order of magnitude increase, concurrent with the injury, may improve the survivability of CHF. In some embodiments, the implantable device may accommodate additional leads that can provide the power, waveform and control to generate stem cells anywhere in the body where they may be needed.

In some implementations, the implantable device may generate stem cells to the myocardium, and with its extra ports, may support the stem cell generating leads (electrical wires connecting pacemaker to tissue) that may generate stem cells to any organ or system in the body that would benefit from them.

In some embodiments, the implantable device may allow for the replacement of cartilage in a damaged knee. A generator in or near a knee in a variety of ways. For example, a generator may be worn on a belt around the leg and a lead that goes to the knee, providing a constant supply of the patient's own stem cells under the kneecap to enable re-growth. In some embodiments, a lead may be sufficient where a patient may have a secondary implanted device that may have the capacity to provide trickle charge. In some aspects, the generator may be completely implantable. In some embodiments, the generator may sit at the patient's bedside, and delivery stem cells to the needed tissue 7×24 for as long as is needed. The patient can be at home, not just in the hospital. To soldiers wounded in the field, this technology can be in the MASH Unit.

For example, the implantable device may generate stem cells for delivery to the knee to rebuild cartilage. The generator with power may be worn on a belt; or strapped above the ankle; and deliver a flow of cells to the area in need. This may not limited to the knee, but may include any area needing tissue repair or regrowth, such as, for example, the spine, the liver, the kidneys, the brain, wherever the stem cells have a desired effect.

In some embodiments, the delivery mechanism may function similarly to an insulin pump with a lead that can be placed into the knee and connected to the monitoring and power device. The monitoring and power device may be worn on a belt or attached to the body during the period of cartilage reconstruction.

Similarly, the stem cell generator can be placed in other areas of the body. By placing a device within a damaged knee, new cartilage can be generated to offset the damage. Similarly new disks can be generated in s spine, and new brain cells generated in a damaged brains, etc.

In some embodiments, a capture mechanism may capture the increased cell generation by tapping into the body to deliver more adult stem cells to the body. In some aspects, the capture mechanism may comprise a collection vessel in the body or external. The stored collected stem cells may be used for a range of treatments, including bathing the myocardium, injection into the myocardium, injection into the Knee, and injection into the spine. In some implementations, infusion into the blood stream may increase stem cell availability to the organism, and infusion into the CellTraffix cell collection system may re-circulate and capture adult stem cells without harm and while keeping them vital.

In some aspects, the captured cells may be held vital in a container that may be permanently or temporarily embedded, accessed as needed, replenished as needed, and delivered into the body into as needed, and delivered to the body for use in subsequent delivery and engrafting. In some aspects, a generator may be paired or integrated with any medical device to provide the ability to generate from nucleated cells dedifferentiated cells and to store and/or deliver them to the needed areas. In some implementations, a capture mechanism that may induce and collect cells either into a flow or into a container, which can be in situ, in vivo, ex vivo, attached to an apheresis device for extraction, implanted, etc.

In some embodiments, an electrode may form the basis for a device that may generate a flow of stem cells on demand, which either flow into the myocardium increasing the normal availability by several hundred fold, or are directed directly into tissue in the myocardium reducing the need for intra-myocardial injections.

In some implementations, an implantable device may be worn on the body, on a crash cart, at a bedside, in a mash unit. The core functionality may be moved among various devices and delivery options. The extra electrodes may serve multiple functions. The logic unit may control device power, circuitry, sensing and input/output. Additional leads may be delivered for pacing, for low voltage defibrillation, for neurostimulation, and for stem cell generation. Each lead may be enabled for or programmed for each or all functions.

In some embodiments, the device may be optionally programmed remotely, such as after a physician's analysis and determination of the rate, position, and type of stem cells needed by the patient. In some aspects, the implantable device may deliver a patient's own stem cells to any needed organ, or extraction for future treatment, etc. with the advantages that the stem cells are their own adult stem cells and will not reject, the volume available can be massively greater than previous methods (bone marrow transplant, fat reduction, umbilical cord extraction, et al) enabled, thus allowing a much improved level of engraftment for any organ needing stem cells.

Additionally, organs such as the heart, which may need a supply at any point in time that the heart suffers a myocardial infarction, can allow for the heart muscle and circulatory system to have an order of magnitude increase in stem cell flow to allow the entire organ to be bathed in an ongoing supply of stem cells until the need is lifted.

There have been many successful examples of stem cell infusion to the myocardium, with tremendous success in rebounding heart health. Stem cells delivered shortly after an MI have been shown to reduce the instances and progression of congestive heart failure (CHF), which can be fatal within five years. Many other organs can benefit from a supply of adult stem cells from a donor. In some implementations, the implantable device may be used in-vivo, in situ, or ex-vivo, it can be permanently or temporarily implanted. It can be part of an EMT or MASH portable device, or on a crash cart in a hospital or doctor's office. It can also be used to extract a supply of adult stem cells to be on hand for future operations and procedures.

### Illustrative Example

The device is designed such that the sample enters via the inlet shown to the left, fills the sample input slot, and then spreads along the surface that will perform the cell selection. As the sample spreads, the bulk of the material flows over the cell harvest slit and exits via the outlet shown on the right. During this process, a very small fraction of the bulk fluid, (enriched with the now rolling stem cells), is diverted into the harvest, which, along with the subsequent drop in shear stress, should allow the cells to dissociate from the selectin surface.

Stem cell generator device can be permanently or temporarily implanted, or stand as an external device attached with apheresis. It can also deal with extracted fluids fed to it from a container. In some embodiments, a high concentration of stem cells may be induced into blood flow and this device harvests them from blood.

The electrical inducer of stem cells is applied to a device that has access to blood, lymph, tissue or other materials. The voltage is applied to the upper container or somewhere in the line so that the fluid gets the electrical charge that induces the increase in conversion of nucleated cells to dedifferentiated cells. These cells are moved to a chamber by a variety of means and held in a container which keeps them vital.

The undifferentiated stem cells can then be moved to an organ by a variety of means, including a catheter to a needle in tissue, or via a flow into a fluid such as blood in which the target organ is bathed, or into lymph, or capillaries positioned for infusion, or any other means. The power to cause differentiation comes from a power source which can be regulated based on a variety of conditions including need, flow.

The components can be combined with the collection and storage device, or can be incorporated into a device such as a pacemaker. A pacemaker, detecting an MI, can turn on the power to the recirculator so it powers the process of dedifferentiation until such time as enough has been generated and delivered, or stored for infusion or extraction, or other needs.

In some embodiments, implantable devices such as pacemakers may be used to send other biomedical energy signals. This can be changing the circuitry to perform this new function, or adding it as a second question, with additional leads to deliver signals. Other biomedical energy signals may include improved RF signals to achieve improved biological impacts, including heating, inflammation reduction, oxygenation, and more. In some aspects, signals for mm-wave energy may be used to treat tumors and improving vascular and tissue healing;

In some implementations, adding secondary function, such as a pacemaker, which also provides a reduction in inflammation, may improve signaling between devices, so that a patient getting a secondary device may be able to gain more than the sum of the parts of functionality by having the two devices communicate. In some aspects, the first device may not be aware of the second device but may have the ability to accommodate any number of future devices, and communicate bi-directionally.

In some aspects, a tumor may be irradiated with RF energy, wherein the tumor reverses and the immune system may learn how to attack cancer cells, not limited to the type of strain of the tumor type. Methods that direct energies may be used to treat other conditions of an organism. In some embodiments, the device can measure, diagnose problems, and plan and execute therapeutic treatments.

Methods in which ultrasound can be modulated to interfere with mitotic spindles of cancer cells during cell division, without interfering with the cell division of healthy cells, resulting in a low toxicity method to treat cancer cells of humans and animals. In some embodiments, these devices may be mounted on robots, such as the DaVinci robot from Intuitive. The adaptation of implantable devices to share information between them patient gains the benefit of multiple sensors and analytics.

In some embodiments, an implantable device may provide anti-inflammatory induction, Faraday Cage effect reduction (chip or mesh outside vessel), elute anti-inflammation treatment, release oxygen superoxygenating tissue or blood, and/or radiate tumor to generate immune system response. In some aspects, resonant seeds or circuits placed near a stent or outside of lumen may avoid clotting exposure.

### CONCLUSION

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. An implantable device for inducing endogenous generation of adult stem cells, the implantable device comprising:
an electrode configured to implant into a portion of a human, wherein the electrode is configured to induce dedifferentiation of nucleated cells;
a power source in electrical communication with the electrode; and
a logic block in logical communication with the electrode and the power source wherein the logic block provides a control signal to fire the electrode in manner conducive to the endogenous generation of adult stem cells.

2. The implantable device of claim 1 additionally comprising a pacemaker device for controlling a rate of heartbeat of a patient into which the implantable device is incorporated.

3. The implantable device of claim 1 further comprising a biometric sensing mechanism in logical communication with the logic block and configured to monitor one or more conditions proximate to an induction site or the implantable device.

4. The implantable device of claim 1, wherein the implantable device comprises an insulin pump.

5. The implantable device of claim 1, wherein the implantable device further comprises a capture mechanism configured to capture and store endogenously generated adult stem cells.

6. The implantable device of claim 1, wherein the implantable device further comprises a delivery mechanism configured to store and deliver endogenously generated adult stem cells to a predefined target.

7. The implantable device of claim 1 wherein the power source comprises an electrochemical battery.

8. The implantable device of claim 1 wherein the power source comprises a solid-state battery.

9. The implantable device of claim 1 wherein the power source comprises a biocell.

10. The implantable device of claim 1 wherein the power source comprises an induction coil.

11. The implantable device of claim 1 wherein the power source comprises an electromagnetic antenna.

12. The implantable device of claim 1 wherein the logic block controls a frequency and duration of electrical stimulation conducive to generating endogenous stem cells.

13. The implantable device of claim 1 wherein the logic block controls a voltage amplitude of electrical stimulation conducive to generating endogenous stem cells.

14. The implantable device of claim 1 wherein the logic block controls a current of electrical stimulation conducive to generating endogenous stem cells.

15. A method for inducing endogenous generation of adult stem cells, wherein the method comprises method steps of providing power to a logic block and operating an implantable device to induce dedifferentiation of nucleated cells in a patient, wherein the implantable device comprises:
the electrode configured to implant into a portion of a human, wherein the electrode is configured to induce dedifferentiation of nucleated cells;
a power source in electrical communication with the electrode; and
a logic block in logical communication with the electrode and the power source wherein the logic block provides a control signal to fire the electrode in manner conducive to the endogenous generation of adult stem cells.

16. The method of claim 15 additionally comprising the step of collecting dedifferented cells generated according to the steps of claim 15.

17. The method of claim 16 additionally comprising the step of administering the collected cells to a predetermined site in a living organism.

18. The method of claim 15 additionally comprising the step of monitoring one or more biometrics of the patient during operation of the implantable device.

19. The method of claim 18 additionally comprising the step of storing an electronic record of a duration of operation of the implantable device in the patient and associating the electronic record with the patient and an administering health care practitioner.

20. The method of claim 15 additionally comprising the step of removing the implantable device from any contact with the patient.
